(19) United States
(12) Patent Application Publication
(10) Pub. No.: US 2003/0018327 A1
(43) Pub. Date: Jan. 23, 2003

(54) Systems and techniques for lung volume reduction

(76) Inventors: Csaba Truckai, Saratoga, CA (US); John H. Shadduck, Tiburon, CA (US)

Correspondence Address: Csaba Truckai, 19566 Arden Court, Saratoga, CA, 95070, US

(21) Appl. No.: 10/198,041

(22) Filed: Jul. 18, 2002

Related U.S. Application Data
(60) Provisional application No. 60/306,749, filed on Jul. 20, 2001.

(51) Int. Cl. A61B 18/14

(52) U.S. Cl. 606/32; 606/41

(57) ABSTRACT

This invention provides least invasive instruments and methods for lung volume reduction. In one embodiment, a catheter has a collapsible working end electrode that can engage an artery wall in a plurality of locations in a patient's bronchial tree to deliver Rf energy to the vessel wall. The application of energy induces the vessel wall to shrink and occlude. The occlusion of the vessel will cause the tertiary bronchus to shrink and wither until cell death is caused wherein the tissue then will be resorbed by the patient's body.

### CROSS-REFERENCE TO RELATED APPLICATION

This application claims benefit of Provisional U.S. Patent Application Ser. No. 60/306,749 filed Jul. 20, 2001 (Docket No. CTX-002) having the same title as this disclosure, which is incorporated herein by reference.

### FIELD OF THE INVENTION

This invention relates to medical systems for accomplishing least invasive techniques for lung volume reduction in a treatment of advanced chronic obstructive lung disease. More particularly, an exemplary system provides a catheter with collapsible working end electrodes that can engage an artery wall in a plurality of locations in a patient's bronchial tree to deliver Rf energy to the vessel wall. The application of energy induces the vessel wall to shrink and occlude thereby causing cell death and resorption of the more distal portion of the brochial tree to reduce lung volume.

### BACKGROUND OF THE INVENTION

Emphysema is a debilitating illness brought about by the destruction of lung tissue. The disorder affects up to 10% of the population over 50 years old. Emphysema is most commonly caused by cigarette smoking and, in some cases, by a genetic deficiency of the enzyme alpha-1-antitrypsin, a protective antiprotease. The condition is characterized by destruction of the alveoli, which are the microscopic air sacs in the lung where gas exchange takes place. Destruction of these air sacs makes it difficult for the body to obtain oxygen and to get rid of carbon dioxide.

In emphysema, there is a progressive decline in respiratory function due to a loss of lung elastic recoil with a decrease of expiratory flow rates. The damage to the microscopic air sacs of the lung results in air-trapping and hyperinflation of the lungs. As the damaged air sacs enlarge, they push on the diaphragm making it more difficult to breathe. The enlarged air sacs also exert compressive forces on undamaged lung tissues, which further reduces gas exchange by the undamaged lung portions. These changes produce the major symptom emphysema patients suffer—dyspnea (shortness of breath) and difficulty of expiration. Current pharmacological treatments for emphysema include bronchodilators to improve airflow. Also, oxygen therapy is used for patients with chronic hypoxemia.

More recently, a surgical procedure called lung volume reduction surgery (LVRS) has been developed to alleviate symptoms of advanced chronic obstructive lung disease that results from emphysema. This surgical resection is variably referred to as lung reduction surgery or reduction pneumoplasty in which the most severely emphysematous lung tissue is resected.

The development of LVRS was based on the observation that emphysema causes the diseased lung to expand and compress the normally functioning lung tissue. If the diseased lung tissue were removed, it was believed that the additional space in the chest cavity would allow the normal lung tissue to expand and carry on gas exchange. LVRS was first introduced in the 1950's but was initially abandoned due to a high operative mortality, primarily due to air leakage. One of the main difficulties of the procedure is suturing the resected lung margin in an airtight manner. Normally there is a vacuum between the ribs and the lungs that helps to make the lungs expand and fill with air when the chest wall expands. If an air leak allows air in the potential space between the ribs and lungs—then the vacuum effect will disappear and the lungs will sag upon chest expansion making it increasingly difficult to inflate the lungs and perform gas exchange.

Currently, there are two principal surgical approaches for LVRS-both of which involve removal of diseased lung tissue (typically in the upper lobes) followed by surgical stapling of the remaining lung to close up the incision. One approach is an open surgery in which the surgeon uses a median sternotomy (MS) to access the chest cavity for removal of diseased lung tissue. The second approach is a video-assisted thoracic surgery (VATS) in which endoscopic instruments are inserted into the chest cavity through small incisions made on either side of the chest. LVRS downsizes the lungs by resecting badly diseased emphysematous tissue that is functionally useless. Surgeons generally remove approximately 20-30% of each lung in a manner that takes advantage of the heterogeneity of emphysema in which the lesions are usually more severe at the apices and less severe at the lung bases. During the course of surgery, one lung is continually ventilated while the lumen of the contralateral lung is clamped. Subsequently, normal areas of the lung deflate as blood flows past the alveoli and resorbs oxygen, while emphysematous portions of the lung with less blood flow and reduced surface area remain inflated and are targeted for resection. The more recent procedures use bovine pericardium or other biocompatible films to buttress a staple line along the resected lung margin to minimize air leaks.

LVRS improves function of the lung by restoring pulmonary elastic recoil and correcting over-distention of the thorax and depression of the diaphragm. Thus, the objective of LVRS is to provide the patient with improved respiratory mechanics and relief from severe shortness of breath upon exertion. Many patients have reported benefits such as improved airflow, increased functional lung capacity and an improved quality of life. As in any major thoracic procedure, there are many risks, including fever, wound infections, wound hematomas, postoperative fatigue and tachycardia. The recuperation period following LVRS varies from person to person, but most patients remain in the hospital for two weeks following surgery. The patient then must endure a regime of physical therapy and rehabilitation for several additional months. Further, the duration of the improvement in lung function following resection is not yet completely known—but there is a suggestion that lung function begins to decline two years after LVRS. Despite optimistic reports, the morbidity, mortality and financial costs associated with LVRS appear to be high, with some studies indicating mortality rates ranging from 4-17%.

### SUMMARY OF THE INVENTION

The present invention provides least invasive instruments and methods for lung volume reduction. In one embodiment, a catheter has a collapsible working end electrode that can engage an artery wall in a plurality of locations in a patient's bronchial tree to deliver Rf energy to the vessel wall. The application of energy induces the vessel wall to shrink and occlude. The occlusion of the vessel will cause the tertiary bronchus to shrink and wither until cell death is caused wherein the tissue then can be resorbed by the patient's body. In a method of using an exemplary system, the physician can advance the catheter endovascularly. In another embodiment, the arteries in the bronchial wall can be damaged and occluded from access via the lumen in the patient's bronchus. Other embodiments utilize coils, glues and hydrogels to occlude a bronchial artery to cause lung volume reduction.

The invention advantageously provides a system and method for least invasive lung volume reduction by occlusion of selected blood vessels that supply the most severely emphysematous lung tissue thus causing the damaged tissue to be resorbed by the patient's body.

The invention provides an endovascular catheter with a working end that carries at least one Rf electrode for shrinking and occluding a targeted site in an artery in a bronchial tree.

The invention provides a method for lung volume reduction (LVR) that is accomplished by an endovascular catheter.

The invention provides a method for LVR that can eliminate the complications of open or endoscopic surgeries.

The invention provides a method for LVR that does not require transection of the exterior lung wall thus eliminating the serious complications of air leakage into the chest cavity.

The invention provides a method for LVR that can greatly reduce the patient's recuperative period and hospital stay.

The invention provides a method for LVR that can be repeated over a patient's lifetime.

The invention provides a method for LVR that will allow for greatly reduced costs when compared to open or endoscopic LVR procedures.

The invention provides a remote electrical source that allows for the delivery of electrical energy to a catheter working end to occlude the blood supply to targeted tertiary bronchial portions.

The invention provides a system with feedback control that modulates power delivery to a catheter working end.

The invention provides a catheter working end and method that utilizes an expandable member with first and second portions of a metallic coating that are adapted to serve as a bi-polar electrode arrangement for occluding a bronchial artery.

### BRIEF DESCRIPTION OF THE DRAWINGS

Other objects and advantages of the present invention will be understood by reference to the following detailed description of the invention when considered in combination with the accompanying Figures, in which like reference numerals are used to identify like components throughout this disclosure.

FIG. 1 shows a schematic view patient's respiratory system and a Type “A” system that comprises an elongate endovascular catheter for lung volume reduction.

FIG. 2A is an enlarged view of the working end of the catheter of FIG. 1 showing an exemplary electrode arrangement deployable from the catheter sleeve.

FIG. 2B is an alternative catheter working end showing an exemplary electrode arrangement carried by a balloon member.

FIG. 3A is a view of the working end of FIG. 2A being deployed in a targeted site in a patient's tertiary bronchus.

FIG. 3B is an enlarged cut-away view of a targeted artery with the working end of FIG. 2A preparing to occlude the vessel.

FIG. 3C is a view similar to FIG. 3B after sealing and occluding the targeted site.

FIG. 4 is an alternative catheter working end shown in a cut-away view of the targeted vessel wherein the working end deploys an occlusive coil.

FIG. 5 is an alternative catheter working end shown in a cut-away view of a targeted vessel wherein the working end deploys a volume of a microporous hydrogel to occlude the vessel.

FIG. 6A is a schematic view of a patient's respiratory system and a Type “B” system of the invention that comprises a member that is adapted for deployment through the patient's bronchus for lung volume reduction.

FIG. 6B is an enlarged view of the working end of the catheter of FIG. 6A showing an exemplary electrode arrangement about a balloon surface that is adapted to perform a method of the invention to occlude blood vessels in the bronchial wall.

### DETAILED DESCRIPTION OF THE INVENTION

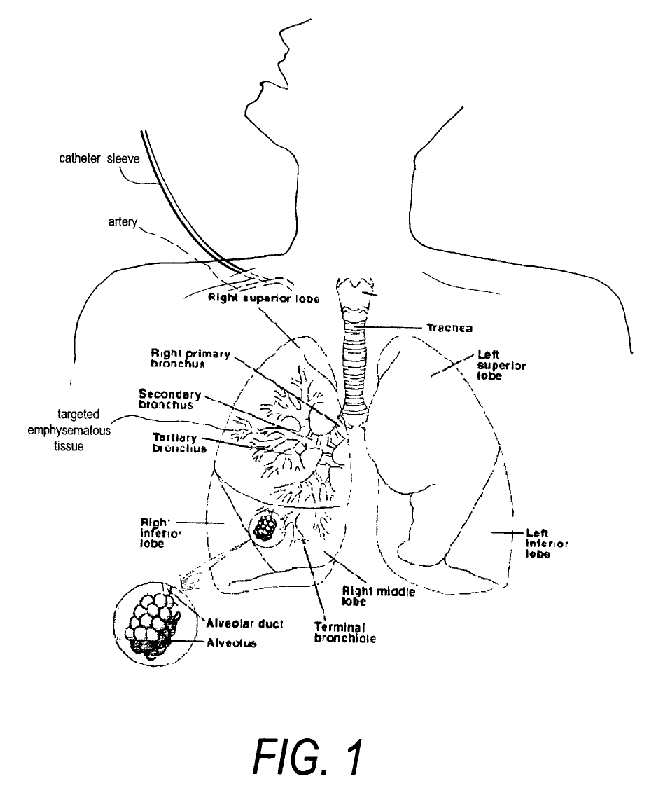

1. Type “A” system for lung volume reduction. FIG. 1 shows a schematic view of a patient's body and lungs 4 with a Type “A” endovascular system 5 introduced from a brachial artery to occlude a targeted site ts in an artery 6 that lies within the wall of the bronchus, which is one of the subdivisions of the trachea that serves to convey air to and from the lungs. The “bronchial tree” as shown in FIG. 1 consists of the primary (right and left) bronchus that branches into the secondary bronchus and tertiary bronchus. In this disclosure, the term tertiary bronchial portions defines any bronchial portions distal to the tertiary branches that lead to terminal bronchioles that may be targeted for reduction.

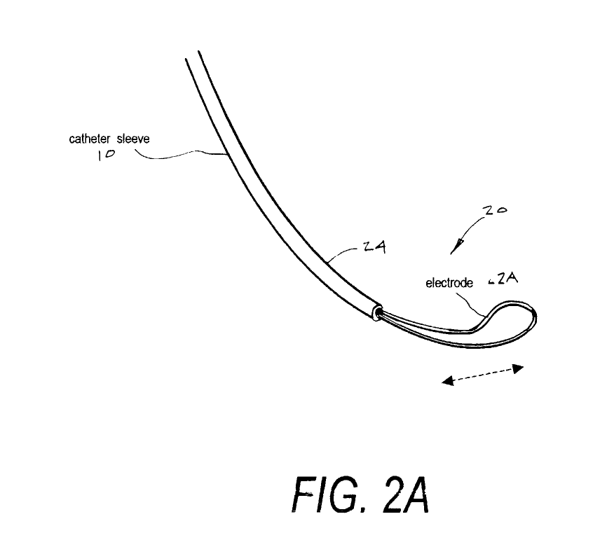

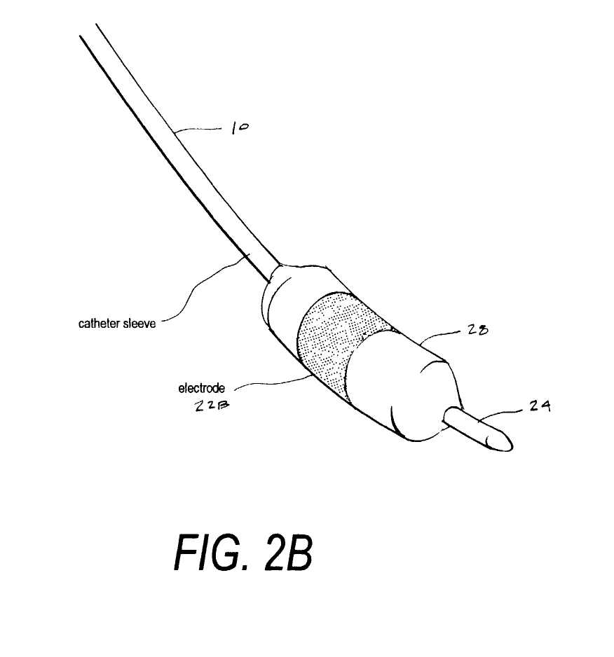

The catheter system has a proximal handle or manifold 9 as is known in the art that is coupled to an elongate microcatheter sleeve 10. FIGS. 2A-2B illustrate enlarged views of exemplary working ends 15 of catheter sleeve 10 that carry an electrode arrangement suitable for engaging the wall of the artery at a targeted site ts. The catheter sleeve 10 can be any suitable diameter, for example, from about 2 Fr. to 6 Fr. In the embodiment of FIG. 2A, the electrode 22A comprises a wire element that is extendable from the distal end 24 of sleeve 10 to assume a slightly expanded cross-sectional dimension compared to the catheter diameter. The electrode wire element 22A is of a shape memory material such as nitinol and preferably can form a loop or any other expanded shape. The proximal end (not shown) of the electrode wire element 22A is coupled to a remote radio-frequency source 25 as is commonly used in electrosurgical applications. In use, the electrode 22A cooperates with a return electrode, such as a ground pad, coupled elsewhere to the patient's body. FIG. 2B illustrates an alternative embodiment of working end that carries electrode 22B in a band about an expandable member 28 such as an inflatable balloon. The purpose of the working end is simply to provide means for substantial engagement of the electrode arrangement with the vessel wall at the targeted site. Therefore, any single electrode or plurality of electrodes (mono-polar or bi-polar) that can be exposed at the working end of the microcatheter 10 falls within the scope of the invention for performing the method described below.

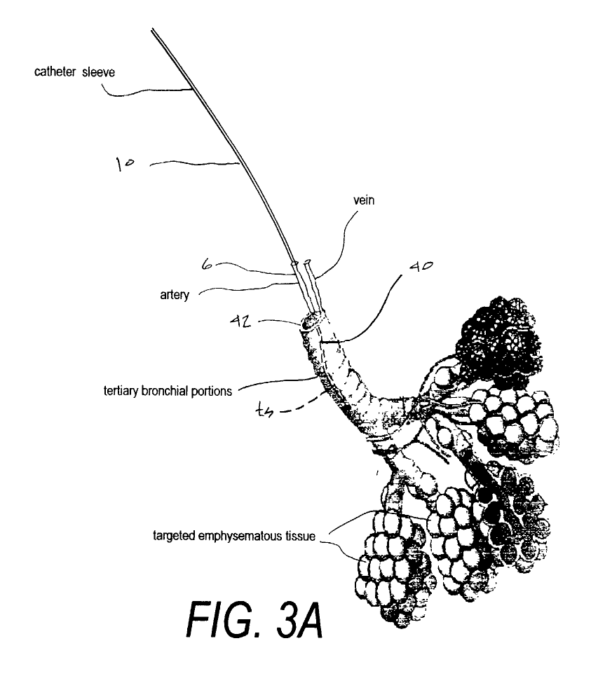

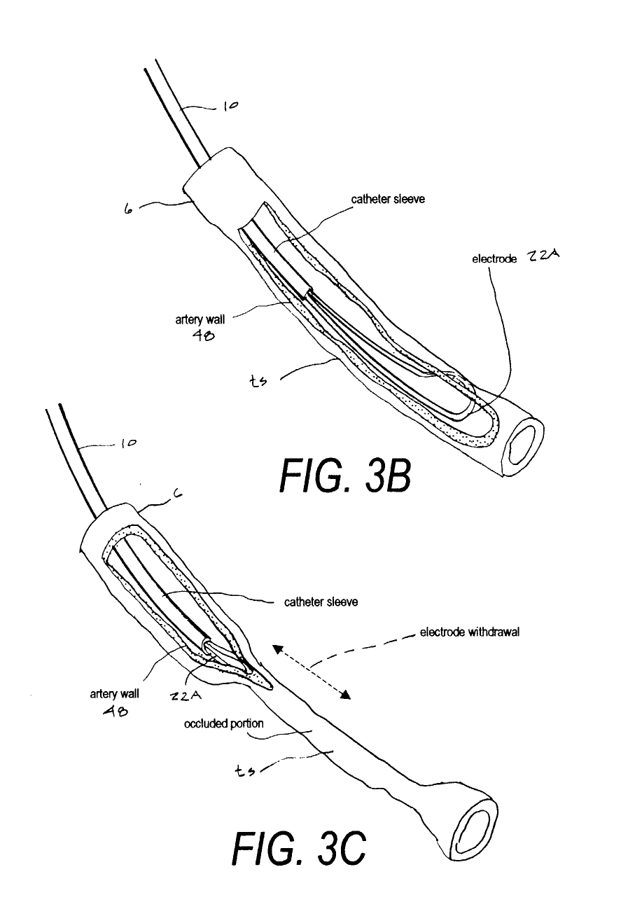

FIGS. 1 & 3A-3C illustrate an exemplary method of utilizing Rf energy to occlude a targeted arterial site ts with the working end 15 of FIG. 2A to accomplish lung volume reduction. FIG. 1 shows the catheter 10 introduced into a brachial artery but any access site is possible. The catheter working end 15 is directed to the targeted site by any imaging means known in the art of endovascular interventions (e.g., ultrasound). FIG. 3A provides a view of the bronchus 40 having a wall 42 that carries an artery 6 in which the targeted site is indicted at ts. The downstream alveoli 44 comprise emphysematous lung tissue which is to be reduced by the method of the invention. FIGS. 3B-3C next shows a cut-away view of the artery 6 alone and the deployment and activation of the Rf electrode loop 22A of FIG. 2A. In FIG. 3B, the collapsible electrode is extended from the working end and its expansion causes it to thereby press outwardly against the vessel walls 48. The delivery of Rf energy to the electrode causes thermal effects in the vessel wall thereby inducing the artery to shrink and occlude. FIG. 3C shows the electrode loop 22A being (optionally) withdrawn proximally into the sleeve 10 while still energized to provide an elongate seal and occlusion of the artery. This method can be repeated at a number of locations to thereby deprive lung tissue downstream from the targeted sites of blood flow. It is believed that the downstream emphysematous tissue will then wither and slowly be resorbed by the body thus resulting in an effective reduction in lung volume by shrinking and resorption of such damaged tissue. Thus, one method of the invention includes any occlusion of targeted sites in arteries that supply tertiary bronchus portions by application of Rf energy thereto from the working end of a microcatheter. It can be appreciated that the expansion member of FIG. 2B and its electrode 22B can be similarly utilized to occlude and seal an artery (not shown).

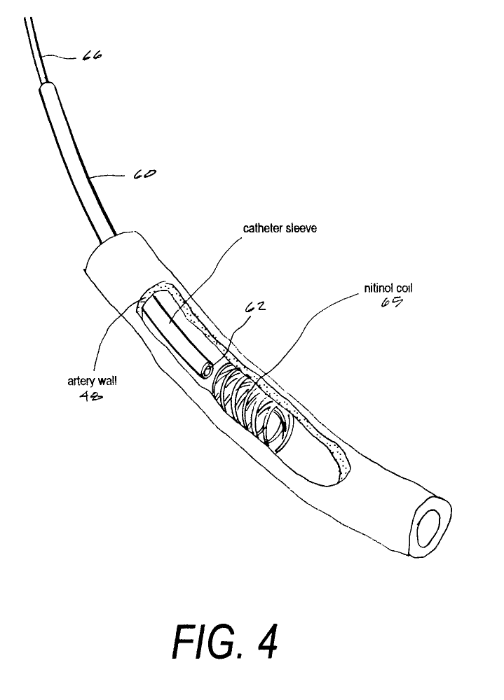

FIG. 4 shows another embodiment of microcatheter sleeve 60 that has a lumen 62 in its working end that carries an occlusion coil 65 that is deployable by a pusher member or mechanism 66. The coil can be of nitinol that is adapted to expand in cross-sectional dimension to engage the vessel wall while at the same time carrying a core of nitinol strands or a polymer film to substantially or completely block blood flow therethrough. Following deployment of the coil 65 at a targeted site, the downstream emphysematous tissue will die and be resorbed by the body to reduce lung volume.

Another embodiment of microcatheter sleeve 80 (not shown) can carry an internal lumen 82 that carries a cyanoacrylate or other similar glue-type biocompatible agent that can be introduced into a patient's blood vessel to occlude the vessel at a targeted site. As described previously, the occlusion can deprive downstream damaged tissue of nourishment causing the dying tissue to be resorbed by the body to reduce lung volume.

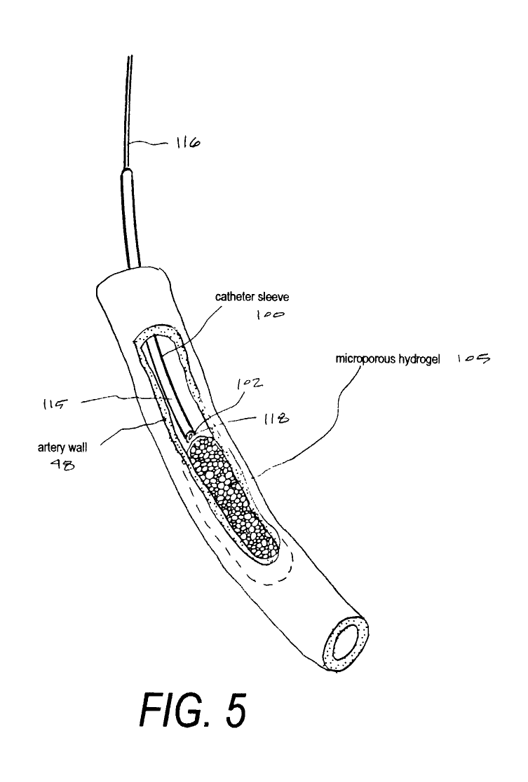

FIG. 5 shows another embodiment of microcatheter sleeve 100 that has an internal lumen 102 that carries a desiccated hydrogel volume 105 that can be deployed into a targeted site in the blood vessel. A microporous or superporous hydrogel is an open cell foam that can be desiccated and collapsed into a thin film or folded and compressed into a suitable form for carrying in the lumen 102 of the catheter. Preferably, the hydrogel is resorbable. The hydrogel volume 105 is deployed from the working end 115 of the catheter by a pusher member 116 that is actuatable from the catheter handle. A fluid-tight film or gel indicated at 118 is carried about the distal end of lumen 102 to substantially prevent fluids from interacting with the hydrogel before its deployment. After deployment from the catheter, exposure of the hydrogel volume 105 to a fluid such as blood will expand the hydrogel to a controlled dimension to engage the walls of the artery. A suitable hydrogel can be any biocompatible fast-response gel, for example of PVME, HPC or the like (see, e.g., S. H. Gehrke, Synthesis, Swelling, Permeability and Applications of Responsive Gels in Responsive Gels, K. Du\check{s}ek (Ed.) Springer-Verlag (1993) pp. 86-143).

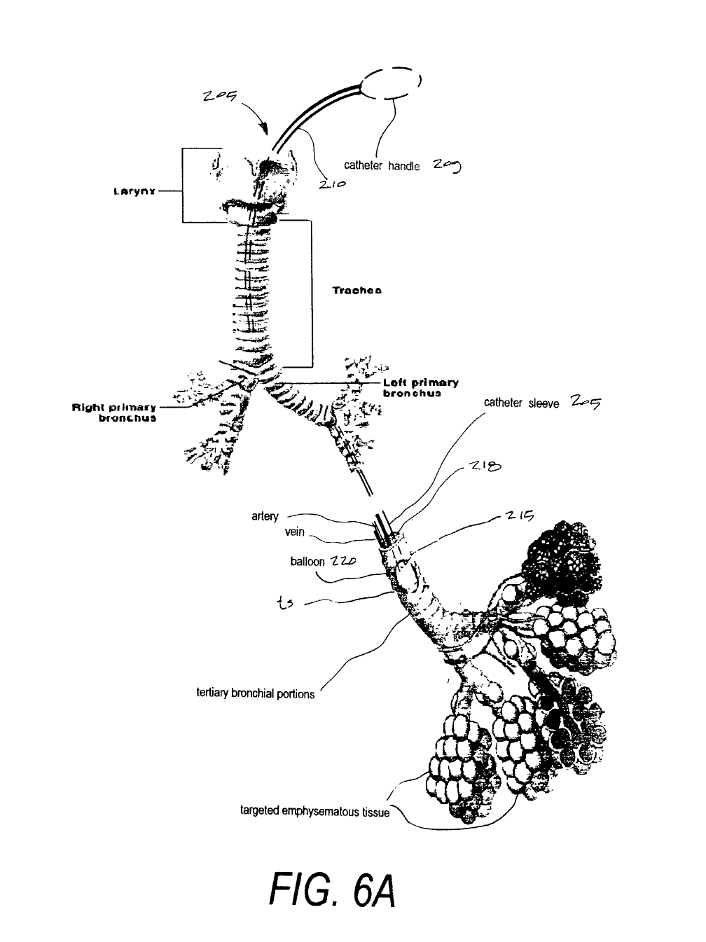

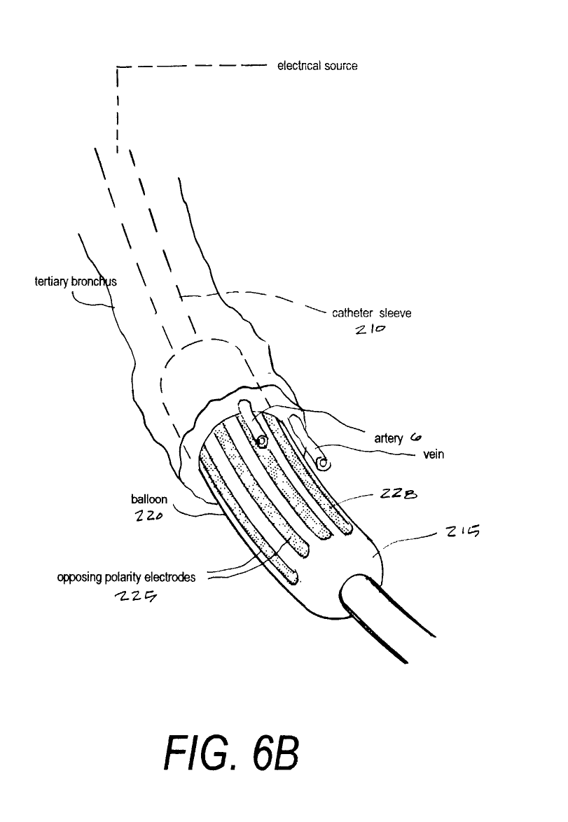

2. Type “B” system for lung volume reduction. FIG. 6A shows another schematic view of a patient's primary and tertiary bronchus with a Type “B” system for LVR that does not comprise an endovascular system—but rather an elongate catheter-type member 205 that is introduced through the patient's bronchial tree to a plurality of targeted sites in the tertiary bronchus. The objective of the system again targets the artery or arteries within the bronchial wall—but this time from a working end 215 positioned within the lumen 218 of a branch of the bronchus.

In one embodiment (FIG. 6A), the elongate member 205 has a proximal handle 209 coupled to an elongate microcatheter sleeve 210. FIG. 6B illustrates an enlarged view of the exemplary working end 215 of sleeve 210 that carries an expandable balloon member indicated at 220. The surface of the balloon 220 carries a plurality of spaced-apart opposing polarity conductor elements 225 (collectively) that are coupled an electrical source to define opposing polarities therein to provide bi-polar Rf energy delivery means. FIG. 6B shows that the balloon can have exposed electrodes of a thin-layer conductive coating 228 that extend axially on the exterior surface of the balloon. The coating 228 can be any suitable biocompatible material that can be deposited on the balloon wall, such as gold, platinum, silver, palladium, tin, titanium, tantalum, copper or combinations or alloys of such metals, or varied layers of such materials. A preferred manner of depositing a metallic coating on the polymer element comprises an electroless plating process known in the art, such as provided by Micro Plating, Inc., 8110 Hawthorne Dr., Erie, Pa. 16509-4654. The thickness of the metallic coating ranges between about 0.0001″ to 0.005″.

In an alternative balloon similar to that of FIG. 6B (not shown), the spaced apart conductor portions 225 can be disposed at least in part helically about the exterior of the balloon. In another balloon embodiment, the spaced apart conductor portions 225 can be disposed at least in part circumferentially about the exterior of the balloon member.

It has been found that some thin coatings of conductive materials, when deposited on an elastomeric balloon, will be stretched and form a series of conductive islands on the balloon exterior which can diminish its functionality as an electrode. A preferred embodiment of balloon member 220 of FIG. 6B has a balloon wall that to provides (i) a first non-elastic wall portion underlying the conductive portions 225, and (ii) a second elastic wall portion in the regions intermediate the spaced apart conductive portions 225. One means for maintaining the first wall portion in a substantially non-elastic condition underlying the outer conductive layer is to provide a suitable layer or web of non-stretch filaments embedded in the balloon wall to prevent its stretching. Alternatively, such flexible but non-stretch filaments can be bonded to either the interior or exterior surface of the balloon underlying the conductive portions 225. As another alternative, any flexible but non-stretchable element, including the electrode itself, can be bonded to a surface of the balloon to prevent localized stretching.

A particular advantage of the balloon 220 described above is that the second elastic balloon wall portion that is intermediate the spaced apart conductive portions 225, upon its expansion to engage the wall of a vessel, will naturally increase the center-to-center distance between spaced apart bi-polar conductive portions 225 which in turn controls the depth to Rf energy delivery and ohmic heating. Thus, the balloon of FIG. 6B will expand to larger cross-sections to engage larger vessels, and at the same time center-to-center spacing between the bi-polar conductors 225 will expand to create deeper ohmic heating in the walls of the larger vessel.

In a method of use, still referring to FIG. 6B, the working end 215 is advanced to the targeted site. An electrical source is actuated to deliver Rf energy to the electrode arrangement 225 to damage and occlude the artery in the wall of the engaged bronchus. The system can further provide at least one feedback control mechanism within a controller for modulating energy delivery to the electrodes. For example, at least one thermocouple can be provided at a surface of the electrode or balloon to measure the temperature of the electrode which is substantially the same as the surface temperature of bronchial wall in contact therewith. The thermocouple is linked to the controller by an electrical lead (not shown). The controller is provided with software and algorithms that are adapted to modulate power delivery from the electrical source to maintain the temperature of the electrodes at a particular level or within a particular temperature range, in response to feedback from the sensor. In a preferred mode of operation, the thermocouple together with feedback circuitry to the controller are used to modulate power delivery to the electrode arrangement 225 to maintain a pre-selected temperature level for a selected period of time. The method of invention maintains the surface temperature within a range of about 60° C. to 100° C. More preferably, the surface temperature of the embolic element is maintained within a range of about 80° C. to 100° C. damage and occlude the blood vessels in the wall.

An alternative embodiment of Type “B” system (not shown) for lung volume reduction can also comprise an catheter member that has a working end that is localizable in the patient's tertiary bronchus with means for accessing the artery in the bronchial wall from the airway lumen. Typically, a needle that is extendable from the catheter working end would be utilized-deployable under intra-operative imaging and guidance (e.g., ultrasound). In this embodiment, the working end then could utilize any of the types of systems described in the Type “A” embodiment to occlude the artery: (i) an electrode arrangement coupled to a remote energy source, (ii) a deployable coil, (iii) an injectable cyanoacrylate, or (iv) a deployable volume of a selected hydrogel.

Those skilled in the art will appreciate that the exemplary embodiments and descriptions of the invention herein are merely illustrative of the invention as a whole. Specific features of the invention may be shown in some figures and not in others, and this is for convenience only and any feature may be combined with another in accordance with the invention. While the principles of the invention have been made clear in the exemplary embodiments, it will be obvious to those skilled in the art that modifications of the structure, arrangement, proportions, elements, and materials may be utilized in the practice of the invention, and otherwise, which are particularly adapted to specific environments and operative requirements without departing from the principles of the invention. The appended claims are intended to cover and embrace any and all such modifications, with the limits only being the true purview, spirit and scope of the invention.

What is claimed is:

1. A method for treating advanced chronic obstructive lung disease by reducing lung volume, comprising the steps of:
(a) introducing a catheter working end endovascularly to at least one targeted site in a bronchial artery;
(b) applying energy to the artery wall at the targeted site by means of electrical current flow from at least one electrode carried at said working end to the artery wall, wherein the application of energy damages and occludes the artery at said targeted site; and
(c) withdrawing the catheter working end from the patient's vasculature wherein the occluded artery causes subsequently causes resorption of portions of the bronchus distal to said targeted site thereby reducing lung volume.

2. The method of claim 1 wherein current flow in step (b) is provided by said at least one electrode functioning with a single polarity.

3. The method of claim 1 wherein current flow in step (b) is provided between first and second spaced apart electrodes functioning with opposing polarities.

4. The method of claim 1 wherein said at least one electrode has a collapsed position and an expanded position and step (a) includes the step of moving the electrode to the expanded position to engage the arterial wall.

5. The method of claim 1 wherein step (b) includes the step of moving said at least one electrode along the arterial wall while delivering electrical current flow thereto.

6. The method of claim 1 wherein said at least one electrode is carried on an expandable balloon and step (a) includes the step of expanding the balloon to move said at least one electrode to engage the arterial wall.

7. The method of claim 6 wherein spaced apart bi-polar electrodes are carried on an expandable balloon and step (a) includes the step of expanding the balloon to thereby engage the arterial wall wherein the degree of expansion of elastic portions of the balloon wall intermediate said bi-polar electrodes controls the center-to-center dimension between said bi-polar electrodes and thereby controls the depth of energy delivery in the engaged arterial wall.

8. A method for treating advanced chronic obstructive lung disease by reducing lung volume, comprising the steps of:
(a) introducing a catheter working end endovascularly to at least one targeted site in a bronchial artery;
(b) deploying an occlusive device from said working end into the lumen of the artery to occlude the artery at said targeted site; and
(c) withdrawing the catheter working end from the patient's vasculature wherein the occluded artery causes subsequent resorption of portions of the bronchus distal to said targeted site thereby reducing lung volume.

9. The method of claim 8 wherein step (b) deploys a self-expanding body of a shape memory material.

10. The method of claim 9 wherein the self-expanding body is of nitinol.

11. A method for treating advanced chronic obstructive lung disease by reducing lung volume, comprising the steps of:
(a) introducing a catheter working end endovascularly to at least one targeted site in a bronchial artery;
(b) deploying a biocompatible glue from said working end into the lumen of the artery to occlude the artery at said targeted site; and
(c) withdrawing the catheter working end from the patient's vasculature wherein the occluded artery causes later resorption of portions of the bronchus distal to said targeted site thereby reducing lung volume.

12. The method of claim 11 wherein the biocompatible glue is cyanoacrylate.

13. A method for treating advanced chronic obstructive lung disease by reducing lung volume, comprising the steps of:
(a) introducing a catheter working end endovascularly to at least one targeted site in a bronchial artery;
(b) deploying a volume of a desiccated hydrogel into the artery from the catheter working end;
(c) wherein hydration and expansion of the hydrogel volume engages the artery walls to occlude the artery at said targeted site; and
(d) withdrawing the catheter working end from the patient's vasculature wherein the occlusion causes subsequent resorption of portions of the bronchus distal to said targeted site thereby reducing lung volume.

14. The method of claim 13 wherein the hydrogel is bioabsorbable.

15. The method of claim 13 wherein the hydrogel is a microporous or superporous gel.

16. A method for treating advanced chronic obstructive lung disease by reducing lung volume, comprising the steps of:
(a) introducing an elongate member through airway in a patient's bronchus to at least one targeted site;
(b) applying energy to said targeted site by delivering electrical current flow at a selected power level to at least one electrode carried at said working end, wherein the application of energy damages and occludes blood vessels in the bronchial wall at said targeted site; and
(c) withdrawing the member wherein the occluded blood vessels cause subsequent resorption of portions of the bronchus distal to said targeted site thereby reducing lung volume.

17. An endovascular catheter system for treating advanced chronic obstructive lung disease by reducing lung volume, comprising:
an elongate flexible catheter sleeve extending along an axis from a proximal handle portion to a working end;
an expandable balloon member carried at the working end;
first and second spaced apart thin-film conductor portions carried on a surface of the expandable balloon;
wherein said first and second conductor portions are operatively coupled to an electrical source defining opposing polarities therein for delivering energy to body structure engaged by said conductor portions; and
wherein said first and second conductor portions are substantially non-elastic portions of the balloon wall with elastic portions of the balloon wall being intermediate said first and second conductor portions.

18. The endovascular catheter system of claim 17 wherein said first and second conductor portions comprise a plurality of axially-extending elements.

19. The endovascular catheter system of claim 17 wherein said first and second conductor portions comprise a plurality of helically-extending elements.

20. The endovascular catheter system of claim 17 wherein said first and second conductor portions comprise a plurality of circumferentially-extending elements.
